(19) United States
(12) Patent Application Publication
(10) Pub. No.: US 2008/0268535 A1
(43) Pub. Date: Oct. 30, 2008

(54) Method for Inducing the Differentiation of Human Myelogenous Leukemia Cells Into Megakarocytes or Thrombocytes

(75) Inventors: Gil-Ja Jhon, Seoul (KR); So-Yeop Han, Seoul (KR); Jin-Kyung Limb, Seoul (KR); Hyun-Jin Cho, Seoul (KR); Shin-Young Kim, Seoul (KR)

Correspondence Address: Casimir Jones, S.C., 440 Science Drive, Suite 203, Madison, WI, 53711, US

(73) Assignee: EWHA University - Industry Collaboration Foundation, Seoul (KR)

(21) Appl. No.: 12/093,381

(22) PCT Filed: Nov. 17, 2006
(86) PCT No.: PCT/KR06/04854

§ 371 Date: Jun. 10, 2008

(30) Foreign Application Priority Data
Nov. 17, 2005 (KR) 10-2005-0110104

(51) Int. Cl. C12N 5/08

(52) U.S. Cl. 435/372

(57) ABSTRACT

Disclosed herein is a method of inducing the differentiation of leukemia cells derived from human bone marrow into megakaryocytes or thrombocytes, comprising the steps of: (a) culturing OP9 cells; (b) layering leukemia cells derived from human bone marrow over the OP9 cells; and (c) incubating the cells in the presence of a compound ((R)-NALPCE) represented by Chemical Formula 1.

### TECHNICAL FIELD

The present invention relates to a method for inducing the differentiation of leukemia cells derived from human bone marrow into megakaryocytes or thrombocytes, comprising (a) culturing OP9 cells; (b) layering leukemia cells derived from human bone marrow on the OP9 cells; and (c) incubating the cells in the presence of the compound of Chemical Formula 1((R)-NALPCE).

### BACKGROUND ART

The studies of the regulation of megakaryocytopoiesis and thrombocytopoiesis have been carried out by Mazur (Exp. Hematol., 15:248, 1987) and Hoffman (Blood, 74:1196-1212, 1989). For example, bone marrow pluripotent stem cells are differentiated into megakaryocytes, erythrocytes, and myelocytes. Megaloblasts are among the megakaryocytic lineage cells detectable in the early stages of development. These cells have basophilic cytoplasms, reticular chromatin, and one morphologically irregular nucleus containing several nucleoli, and range in diameter from 20 to 30 μm. Within a short time, megakaryocytes have up to 32 nuclei (polyploidy) while the cytoplasm remains largely immature. With the advance of maturation, the nuclei undergo further lobulation and concentration while the cytoplasm increases in volume and is further acidophilic and granulated. In the most mature megakaryocytic lineage cells, platelets are observed to be released from cell verges. Generally, less than 10% of megakaryocytes are in an erythroblastic stage, while more than 50% undergo maturation. Typically, megakaryocytes are morphologically classified into early-stage progenitor megakaryocytes, mid-stage promegakaryocytes or basophilic megakaryocytes, and late-stage mature megakaryocytes (acidophilic, granulate and responsible for platelet biogenesis). Mature megakaryocytes shed cytoplasmic filaments into sinusoidal lumens wherein they are fragmented into individual platelets (Williams et al., Hematology, 1972).

Platelets, playing a crucial role in hemostasis or blood coagulation, measure 2 to 3 μm in diameter, with a mean blood concentration of 300,000 to 500,000 cells/mm². They are sticky and viscous, and the morphology thereof varies depending on conditions. Platelet depletion is likely to give rise to hemorrhaging. Thrombocytopenia is particularly problematic.

Despite the recent great advances in scientific and medical technology, new incurable diseases and adult diseases have tended to increase in incidence due to various causes, including living environments and diet habits. The incidence of cancer, ranking as the number one cause of death in Korea, increases every year. Chemical therapy and radiotherapy, both applied in the treatment of blood cancer, destroy not only cancer cells, but also bone marrow cells, especially hematopoietic cells, which are responsible for making blood and regulating immune functions. Thus, these therapies suffer from disadvantages of producing serious side effects, such as the prevention of hematopoiesis and the destruction of immunity. For example, the depletion of leukocytes and platelets due to serious damage to bone marrow cells gives rise to the collapse of the immune system, leading to death. In connection with therapy for blood-related cancer, such as leukemia, aplastic anemia, congenital intermittent leukopenia, the absence of congenital immune and hematopoietic progenitor cells, malignant blood diseases, etc., therefore, there is a desperate need for a novel material that can promote the development of hematopoietic progenitor cells, thereby enhancing hematopoietic and immune functions.

Leading to the present invention, intensive and thorough research into hematopoietic and immune functions of blood cells, conducted by the present inventors, resulted in the finding that the compound ((R)-NALPCE) represented by the following Chemical Formula 1, in cooperation with OP9 cells, is highly effective in inducing the differentiation of leukemia cells derived from human bone marrow into megakaryocytes and thrombocytes.

### DISCLOSURE OF THE INVENTION

It is therefore an object of the present invention to provide a method for inducing the differentiation of leukemia cells derived from human bone marrow into megakaryocytes or thrombocytes, comprising the steps of: (a) culturing OP9 cells; (b) layering leukemia cells derived from human bone marrow over the OP9 cells; and (c) incubating the cells in the presence of a compound represented by the following Chemical Formula 1, and a composition for effecting the method.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a graph showing the cytotoxicity of (R)-NALPCE in leukemia cells derived from human bone marrow;

FIG. 2 is a graph showing the growth curves of leukemia cells derived from human bone marrow(K562 cells) in the absence and presence of (R)-NALPCE;

FIG. 3 is a graph showing the growth curves of leukemia cells derived from human bone marrow (HEL cells) in the absence and presence of (R)-NALPCE;

FIG. 4 is a graph showing the expression levels of CD41, CD16, glycophorin A and CD14 receptors in the leukemia cells derived from human bone marrow (K562 cells) treated with (R)-NALPCE and PMA, separately;

FIG. 5 is a graph showing the expression levels of CD61, CD235a, CD16 and CD14 receptors on leukemia cells derived from human bone marrow (HEL cells) treated separately with (R)-NALPCE and PMA;

FIG. 6 provides graphs showing expression levels of CD41 (A) and CD61 (B) in the time-dependent manner according to the (R)-NALPCE-induced megakaryocyte differentiation of leukemia cells derived from human bone marrow (K562 cells);

FIG. 7 provides graphs showing expression levels of CD41 (A) and CD61 (B) in the time-dependent according to the (R)-NALPCE-induced megakaryocyte differentiation of leukemia cells derived from human bone marrow (HEL cells);

FIG. 8 provides graphs showing time-dependent expression levels of CD41 and CD61 according to the megakaryocyte differentiation of the leukemia cells derived from human bone marrow treated with (R)-NALPCE, alone or in coculture system using OP9 cells;

FIG. 9 provides photographs, taken with an optical microscope, comparing the ability to induce the differentiation of leukemia cells derived from human bone marrow (K562 cells) into megakaryocytes with (R)-NALPCE and PMA;

FIG. 10 provides photographs, taken with an optical microscope, comparing the ability to induce the megakaryocytes differentiation of leukemia cells derived from human bone marrow (HEL cells) treated with (R)-NALPCE, PMA and TPO, separately;

FIG. 11 provides photographs, taken with an optical microscope, showing thrombopoiesis in the time-dependent manner in the presence of OP9 cells in megakaryocytes, differentiated from leukemia cells derived from human bone marrow (K562 cells)treated with (R)-NALPCE to thrombocytes;

FIG. 12 is a graph showing the binding effect of fibrinogen and glycoprotein b/a complex of the megakaryocytes differentiated from leukemia cells derived from human bone marrow (K562 cells) treated with (R)-NALPCE in the presence of OP9 cells;

FIG. 13 provides photographs, taken with an electron microscope, showing the megakaryocytes and thrombocytes differentiated from K562 cells treated with R)-NALPCE on the OP9 cells;

FIG. 14 provides photographs, taken with a fluorescence microscope, showing active blood platelets and active platelet-like cells released from the megakaryocytes which are differentiated from K562 cells treated with (R)-NALPCE in the presence of OP9 cells.

### BEST MODE FOR CARRYING OUT THE INVENTION

In accordance with an aspect, the present invention pertains to a method for inducing the differentiation of leukemia cells derived from human bone marrow into megakaryocytes or thrombocytes, comprising (a) culturing OP9 cells; (b) layering leukemia cells derived from human bone marrow on OP9 cells; and (c) adding the compound represented by the following Chemical Formula 1.

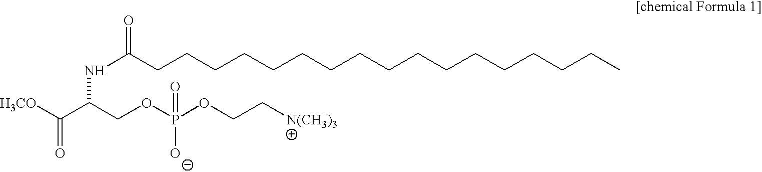

OP9 cells, a kind of stromal cells, lack M-CSF (Macrophage-Colony stimulating factor) and CSF-1 (Colony Stimulating factor-1), which are needed for differentiating embryonic stem cells into cells of various lineages, but express hematopoietic factors, such as SCF (Stem Cell Factor) and IL-6. Cells derived from F2(C57BL/6×C3H)-op/op rat nascent calvaria cells are OP9 cells.

As used herein, the term “leukemia cells derived from human bone marrow” means blood stem cells, that is, progenitor cells capable of differentiating into bone marrow stem cells. These cells can be differentiated into monocytes, neutrophils, eosinophils, erythrocytes, macrophages, megakaryocytes, and thrombocytes. In an embodiment of the present invention, leukemia cells derived from human bone marrow are K562 cells or HEL cells.

By the term “differentiation” as used herein, it is meant that an increased amount of megakaryocytes capable of producing thrombocytes is derived from leukemia cells derived from human bone marrow.

The compound of Chemical Formula 1 according to the present invention (hereinafter referred to as “(R)-NALPCE”) is N-steroyl-O-phosphocholine-D-serine methyl ester, the synthesis method of which is elucidated in detail in Korean Pat. 10-0398892.

There are known many inducers of differentiation of leukemia cells derived from human bone marrow, including PMA (phorbol 12-myristate 13-acetate), TPA (12-O-tetradecanoylphorbol-13-acetate), phorbol dibutyrate, ganglioside GM 3 and (R)-NALPCE. Particularly, PMA is known to be involved in the early stages of the differentiation of leukemia cells derived from human bone marrow into megakaryoblasts, and is most widely used. Comparison of PMA and (R)-NALPCE for the ability to differentiate leukemia cells derived from human bone marrow into megakaryocytes revealed that cells expressing CD41, a receptor specific for megakaryocytes, increased in number more in a medium containing PMA than in a medium containing (R)-NALPCE. However, when (R)-NALPCE is used in combination with OP9 cells, the expression rate of the megakaryocyte-specific receptor CD41 or CD61-positive cells is found to increase by 5 to 15%, compared to when (R)-NALPCE is used alone. This expression level was comparable with the differentiation inductivity of PMA. Particularly, whereas the megakaryocytes, differentiated by PMA treatment, could not secrete thrombocytes, the megakaryocytes differentiated by the induction according to the present invention released a lot of thrombocytes. Further, these released thrombocytes were observed to be morphologically and functionally similar to pre-existing thrombocytes in the blood.

The data obtained in the comparison study indicate that the use of OP9 cells and (R)-NALPCE in combination exhibits differentiation inductivity that is as good as that of PMA, a widely known inducer for differentiating leukemia cells derived from human bone marrow, such as K562 cells or HEL cells, into megakaryocytes. In addition, when being differentiated by the induction in accordance with the method of the present invention, the megakaryocytes, in contrast to those differentiated by PMA induction, are able to secrete thrombocytes, which have properties similar to those of thrombocytes pre-existing in the blood, so that the present invention can be applied for treating blood-related diseases, such as thrombocytopenia.

In an embodiment, the present invention provides a method for inducing the differentiation of leukemia cells derived from human bone marrow into megakaryocytes or thrombocytes, comprising (a) culturing OP9 cells; (b) layering leukemia cells derived from human bone marrow on the OP9 cells; and (c) adding a compound represented by the following Chemical Formula 1 into the cells.

In step (a) of the method, OP9 cells are uniformly spread over a culture dish. In detail, OP9 cells may be placed at a concentration of 1×10³ to 1×10⁷ cells into a 40-mm well, and preferably at a concentration of 1×10⁴ to 1×10⁶ cells per well in a 6-well culture dish, followed by culturing them for 18 to 36 hrs, and preferably for 20 to 28 hrs, to uniformly attach OP9 cells to the bottom.

In step (b) of the method, leukemia cells derived from human bone marrow are layered on the uniformly attached OP9 cells in the culture dish. Preferably, the leukemia cells derived from human bone marrow are K562 cells or HEL cells.

In step (c) of the method, (R)-NALPCE is added onto the leukemia cells derived from human bone marrow. (R)-NALPCE is used at a concentration from 5 to 60 μg/ml, and preferably at a concentration from 10 to 50 μg/ml.

In accordance with another aspect, the present invention pertains to a composition for inducing the differentiation of leukemia cells derived from human bone marrow into megakaryocytes or thrombocytes, comprising OP9 cells and (R)-NALPCE.

The thrombocytes released from the megakaryocytes, which have been differentiated by induction, according to the method of the present invention, can be used in the treatment of patients with thrombocytopenia.

Therefore, the method of the present invention is therapeutically useful for thrombocytopenia, whether it results from the reduction of thrombocyte production due to leukemia, metastatic cancer, blood and bone marrow diseases (e.g., aplitic anemia, primary myelofibrosis, myelodysplasia, etc.), vitamin 12 or folate deficiency, and/or bone marrow injury; the destruction of thrombocytes due to sepsis, valvular heart surgery, systemic lupus erythematosus (S.L.E.), lymphoma, chronic lymphocytic leukemia, infectious diseases (e.g., infectious mononucleosis, etc.), and/or drugs (e.g., penicillin, cephalosporin, thiazide, etc.); or abnormal thrombocyte distribution due to tumor- or portal hypertension-caused splenomegaly.

A better understanding of the present invention may be obtained in light of the following examples which are set forth to illustrate, but are not to be construed to limit, the present invention.

### EXPERIMENTAL EXAMPLE 1

### Preparation of Compound of Chemical Formula 1 ((R)-NALPCE)

1-1. Synthesis of D-serine methyl ester hydrochloric acid salt

A solution of 47.7 mmol of D-serine in 476 ml of serine methanol was saturated with hydrochloric acid gas and allowed to react at room temperature for 2 hrs. Following the evaporation of the solvent, recrystallization with methanol and ether produced the object compound L-serine methyl ester hydrochloric acid salt (Yield: 99%, m.p.: 163-164° C., [a]²⁵_{D}=−4.3 (c 1.8, EtOH)). The structure of the synthesized compound was identified using FTIR, ¹H-NMR and ¹³C-NMR.

FTIR (KBr, cm⁻¹): 3349 O—H peak, 2943 sp³ C—H peak, 1749 ester carbonyl peak

¹H NMR(CD₃OD): δ4.07˜4.10 (1H, t, J=3.9 Hz), 3.38-3.93 (2H, m), 3.79 (3H, s) methoxy carbon cation (s: singlet, d: doublet, t: triplet, m: multiplet)

¹³C NMR(CD₃OD): δ52.69, 55.10, 59.67, 168.37 carbonyl peak

1-2. Synthesis of N-steroyl-D-serine methyl ester

The compound (1 eq) synthesized in 1-1 was dissolved in 257 ml of dichloromethane and cooled to 0° C. To this solution were sequentially added N-methyl morpholine (2.1 eq), stearic acid (1.1 eq) and 1-hydroxybenzotriazole (1.1 eq), 1,3-dicyclohexylcarbodiimide (1.1 eq) in that order, and the reaction was conducted for 1 hr, and then for 3 hrs at room temperature. Following the completion of the reaction, the by-product dicyclourea was filtered off in a vacuum and the remaining filtrate was concentrated. The concentrate was purified using column chromatography (dichloromethane:acetone=9:1→7:1) to afford the object compound N-steroyl-D-serine methyl ester (Yield: 88%, m.p.: 82-83° C., [a]²⁵_{D}=−15.7 (c 2.0, CHCl₃)). The synthesized compound was identified by structural analysis through FTIR, ¹H-NMR and ¹³C-NMR.

FTIR (KBr, cm⁻¹): 3310 O—H peak, 2919 sp³ C—H peak, 1720 ester carbonyl peak, 1650 amide carbonyl peak

¹³H NMR(CDCl₃): δ0.83˜0.88 (3H, m) stearic acid terminal carbon cation, 1.23 (28H, s) hydrocarbon cation, 1.60˜1.63 (2H, m) carbonyl-β-carbon cation, 2.21˜2.28 (2H, t, J=7.6 Hz), 2.52 (1H, m) hydroxyl group peak, 3.78 (3H, s) methoxy carbon cation, 3.93˜3.94 (2H, d, J=3.4 Hz), 4.64˜4.70 (1H, m), 6.36˜6.39 (1H, d, J=6.5 Hz) amide nitrogen cation

¹³C NMR(CDCl₃): δ14.1 stearic acid terminal carbon, 22.7 hydrocarbon carbon, 25.5 carbonyl-β-carbon, 29.2, 29.3, 29.5, 29.7, 31.9 hydrocarbon, 36.5, 52.8 methoxy carbon, 54.6, 63.7, 171.0 carbonyl peak, 173.8 carbonyl peak

1-3. Synthesis of N-steroyl-O-phosphocholine-D-serine methyl ester

A solution of the compound (1 eq) synthesized in 1-2 in 260 ml of tetrahydrofuran was cooled to −10° C. To the solution were added N-diisopropylethylamine (4 eq) and ethylenechlorophosphite (3 eq), followed by reaction for 1 hr. The addition of bromine (3 eq) and reaction for 15 min was conducted before the addition of 86.6 ml of water and reaction for 1 hr at room temperature. The organic layer thus separated was evaporated, followed by recrystallization in dichloromethane and acetone. The precipitate was re-dissolved in 87.5 ml of chloroform/isopropanol/acetonitrile (3:5:5, v/v/v) at 0° C., and 40% aqueous trimethyl amine (3 eq) was added to this solution before reaction for 11 hrs. Purification through column chromatography (dichloromethane: methanol:water=3:1:0→2:1:0.1) afforded the object compound N-steroyl-O-phosphocholine-D-serine methyl ester (Yield: 12%, [a]²⁵_{D}=+8.8 (c 2.0, MeOH)). The synthesized compound was identified by structural analysis through ¹H-NMR and ¹³C-NMR.

¹H NMR(CDCl₃): δ0.90˜0.93 (3H, m) stearic acid terminal carbon cation, 1.31 (28H, s) hydrocarbon cation, 1.63˜1.65 (2H, m) carbonyl-β-carbon cation, 2.27˜2.33 (2H, t, J=7.2 Hz), 3.25 (9H, s) trimethylamine carbon cation, 3.65˜3.67 (2H, m), 3.77 (3H, s) methoxy peak, 4.15˜4.19 (1H, m), 4.21˜4.28 (3H, m), 4.68 (1H, m);

¹³C NMR(CDCl₃): δ13.5 stearic acid terminal carbon, 22.8 hydrocarbon carbon, 25.9 carbonyl-β-carbon, 29.3, 29.5, 29.8, 32.1, 35.7, 51.9 methoxy carbon, 53.7, 59.5, 65.1, 66.4, 170.6 carbonyl peak, 175.4 carbonyl peak

### EXPERIMENTAL EXAMPLE 2

### Cytotoxicity Assay of (R)-NALPCE

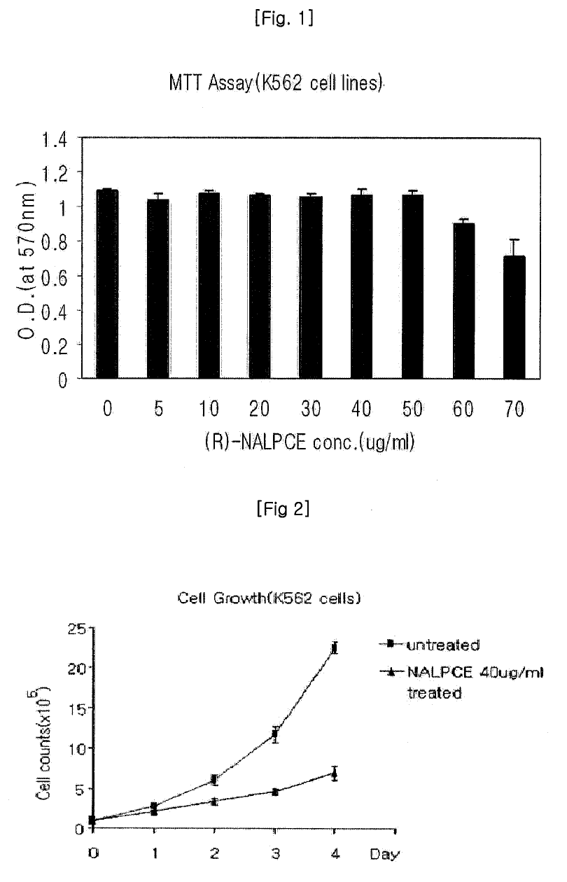

In order to examine whether the compound (R)-NALPCE is toxic to cells, MTT (3-[4,5-dimethylthiazol-2-yl]-2,5-diphenyltetrazolium bromide) assay was conducted in K562 cells which are the leukemia cells derived from human bone marrow. These cells were aliquoted at a density of 4×10⁴ cells per well in 96-well plates and treated with various concentrations of (R)-NALPCE, followed by incubation for 48 hrs. Thereafter, an MTT solution was added in an amount of 20 μl to each well. Incubation at 37° C. for 4 hrs, treatment with a 20% sodium dodecyl sulfate (SDS) solution (in an equivolume mixture of N,N-dimethylformamide and distilled water), and incubation at 37° C. for 16 hrs were sequentially conducted in that order before the absorbance at 570 nm was read using an ELISA reader (Bio-Tek Instrument, Winiiski, Vt.). The assay was conducted in triplicate. The results are given in FIG. 1.

As depicted in FIG. 1, the compound (R)-NALPCE does not exert cytotoxicity on K562 cells, which are the leukemia cells derived from human bone marrow, to the dose of 10 to 50 μg/ml.

### EXPERIMENTAL EXAMPLE 3

### Cell Growth Curve in the Presence of (R)-NALPCE

3-1. Growth Curve of K562 Cells

To examine the effect of the compound (R)-NALPCE on cell growth, K562 cells was monitored for growth in the presence thereof. The cells were aliquoted at a density of 1×10⁵ cells per well in 6-well plates and treated with 40 μg/ml. After staining with trypan blue, viable cells were counted every day for 5 days. The results are given in FIG. 2.

When commencing differentiation into megakaryocytes, cells exhibit a growth curve different from usual. In contrast to cytokinesis, which occurs with cell proliferation, differentiation into megakaryocytes shows asynchronous nuclear-cytoplasmic maturation: mitosis is processed while cytokinesisis blocked, so that a relatively low cell growth curve is plotted.

3-2: Cell Growth Curve in the Presence of (R)-NALPCE

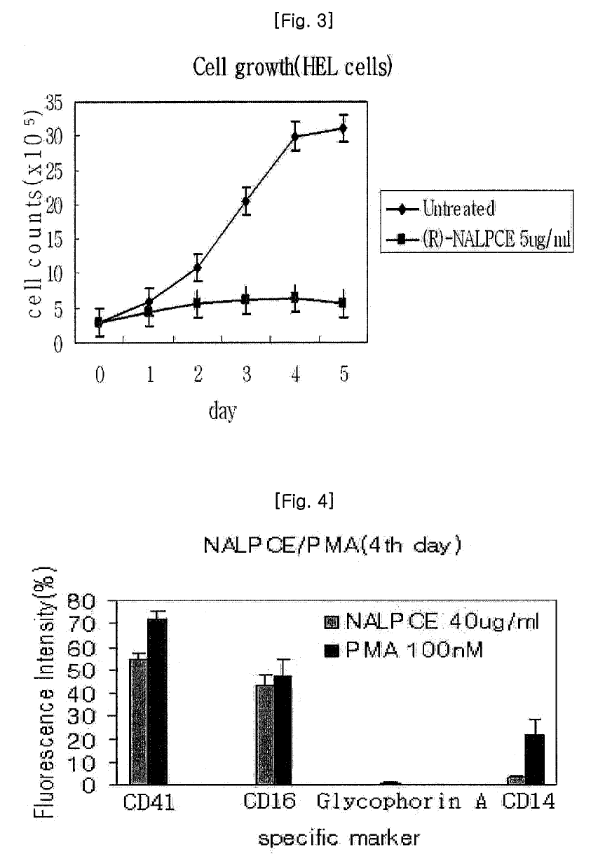

To examine the effect of the compound (R)-NALPCE on cell growth, the HEL cells was monitored for growth curve in the presence thereof. The cells were aliquoted at a density of 3×10⁵ cells per well in 6-well plates and treated with 5 μg/ml of (R)-NALPCE. After staining with trypan blue, viable cells were counted every day for 5 days. The results are given in FIG. 3.

After commencement of differentiation into megakaryocytes, cells did not go through the same growth process as usual, showing an unusual growth curve. In contrast to cytokinesis, which occurs with cell proliferation, differentiation into megakaryocytes shows asynchronous nuclear-cytoplasmic maturation: mitosis is processed while cytokinesisis blocked, so that a relatively low cell growth curve is plotted.

### EXPERIMENTAL EXAMPLE 4

### Comparison of (R)-NALPCE and PMA for Differentiation Inductivity

4-1. Differentiation of K562 Cells

K562 cells were aliquoted at a density of 2×10⁵ per well in 6-well plates and treated with 40 μg/ml of (R)-NALPCE or 30 nM of PMA (12-phorbol myristate-13-acetate, Calbiochem.), followed by incubation for 4 days. After centrifugation (1,000 rpm, 10 min), the cell pellets thus obtained were washed three times with 0.01% bovine serum albumin (BSA) in PBS. The cells were incubated with 10 μl of anti-CD41-fluorescein-5-isothiocyanate (FITC) (Dako Chemicals, Carpenteria, Calif.), 20 μl of anti-CD16-Cy-Chrome (Cy) (BD Pharmingen), 10 μl of anti-glycophorin ACD235a-phycoerythrin (PE) (Dako Chemicals, Carpenteria, Calif.), and 20 μl of anti-CD14-allophycocyanin (APC) (BD Pharmingen) in 200 μl of 0.01% bovine serum albumin (BSA)-PBS at 4° C. for 50 min while spinning to realize homogeneous fluorescent staining. After centrifugation (1,000 rpm, 10 min), the cell pellets thus obtained were washed three times with 0.01% bovine serum albumin (BSA) in PBS. Fluorescence was analyzed on a fluorescence-activated cell sorter (FACS) (Aria) using a Cellquest program. The results are given in FIG. 4.

As seen in FIG. 4, CD41 (megakaryocyte-specific receptor), CD16 (neutrophil-specific receptor), CD14 (monocyte-specific receptor) were expressed, particularly with a great increase of CD41, upon treatment with PMA. In addition, CD41 and CD16 were specifically expressed on the K562 cells treated with (R)-NALPCE.

4-2: Differentiation of HEL Cells

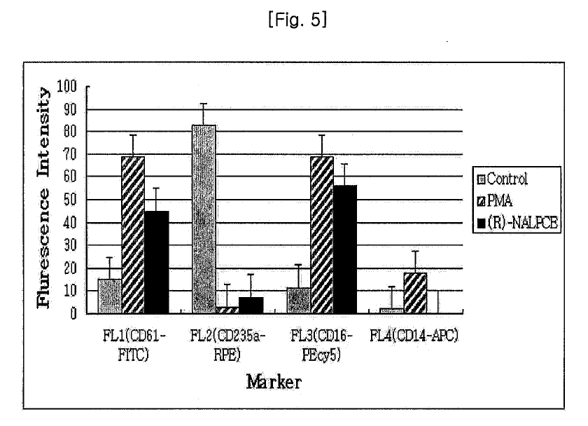

HEL cells were aliquoted at a density of 3×10⁵ cells per well in 6-well plates and treated separately with 5 μg/ml of (R)-NALPCE and 10 nM of 12-phorbol myristate-13-acetate (PMA) (Calbiochem.), followed by incubation for 5 and 3 days, respectively. After centrifugation (1,000 rpm, 10 min), cell pellets thus obtained were washed three times with 0.01% bovine serum albumin (BSA) in PBS and incubated with 10 μl of anti-CD61-fluorescein-5-isothiocyanate (FITC) (Dako Chemicals, Carpenteria, Calif.), 20 μl of anti-CD16-Cy-Chrome (Cy) (BD Pharmingen), 10 μl of anti-glycophorin ACD235a-phycoerythrin (PE) (Dako Chemicals, Carpenteria, Calif.), and 20 μl of anti-CD14-allophycocyanin (APC) (BD Pharmingen) in 200 μl of 0.01% bovine serum albumin (BSA)-PBS at 4° C. for 50 min with spinning so as to achieve homogenous fluorescent staining over the cells. After centrifugation (1,000 rpm, 10 min), the cell pellets thus obtained were washed three times with 0.01% bovine serum albumin (BSA) in PBS. Fluorescence was analyzed on a fluorescence-activated cell sorter (FACS) (Aria) using a Cellquest program. The results are given in FIG. 5.

As seen in FIG. 4, CD61 (megakaryocyte-specific receptor), CD16 (neutrophil-specific receptor), and CD14 (monocyte-specific receptor) were expressed, particularly with a great increase of CD61, upon treatment with PMA. In addition, CD61 and CD16 were specifically expressed on the HEL cells treated with (R)-NALPCE.

### EXPERIMENTAL EXAMPLE 5

### By (R)-NALPCE-Induced Megakaryocyte Differentiation—the Use of Receptor Specific for Megakaryocytes

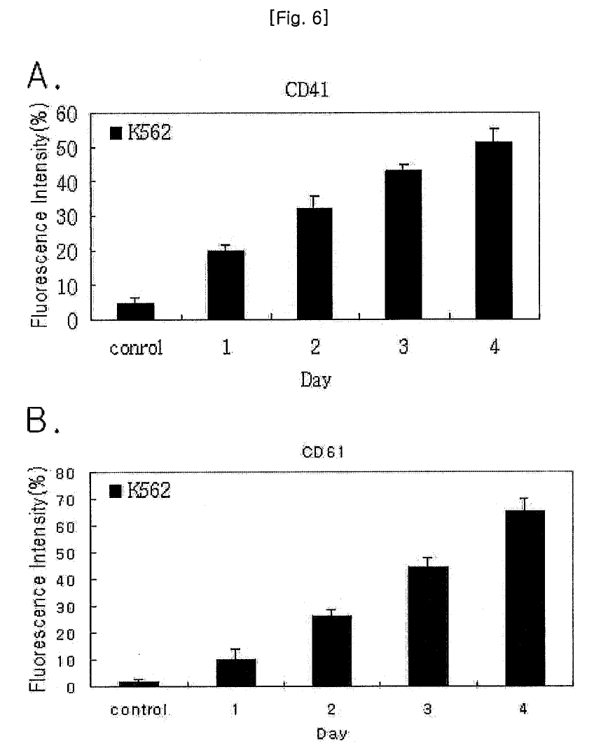

K562 cells were aliquoted at a density of 2×10⁵ cells per well in 6-well plates and treated with 40 μg/ml of (R)-NALPCE, followed by incubation for 4 days. After centrifugation (1,000 rpm, 5 min), the cell pellets thus obtained were washed three times with 0.01% bovine serum albumin (BSA) in PBS. Incubation with 10 μl of anti-CD41-fluorescein-5-isothiocyanate (FITC) (Dako Chemicals, Carpenteria, Calif.) or 20 μl of anti-CD61-phycoerythrin (PE) (BD Pharmingen) in 200 μl of 0.01% bovine serum albumin (BSA)-PBS was conducted at 4° C. for 50 min with spinning to realize homogeneous fluorescent staining. Following centrifugation (1,000 rpm, 10 min), the cell pellets thus obtained were rinsed three times with 0.01% bovine serum albumin (BSA) in PBS. Fluorescence was analyzed on a fluorescence-activated cell sorter (FACS) (Aria) using a Cellquest program. The results are given in FIGS. 6A and 6B.

CD41 and CD61, which both are receptors specifically expressed upon the differentiation of K562 cells into megakaryocytes, were observed to increase in expression rate in a time-dependent manner upon treatment with (R)-NALPCE.

### EXPERIMENTAL EXAMPLE 6

### Differentiation into Megakaryocyte by Induction of (R)-NALPCE—the Use of Receptor Specific for Megakaryocyte

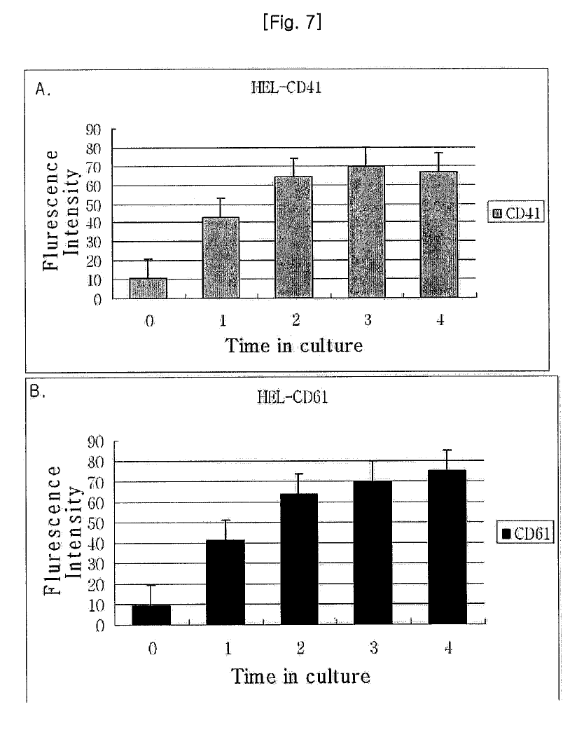

HEL cells were aliquoted at a density of 3×10⁵ cells per well in 6-well plates and treated with 5 μg/ml of (R)-NALPCE, followed by incubation for 5 days. After centrifugation (1,000 rpm, 5 min), the cell pellets thus obtained were washed three times with 0.01% bovine serum albumin (BSA) in PBS. The cells were incubated with 10 μl of anti-CD41-fluorescein-5-isothiocyanate (FITC) (Dako Chemicals, Carpenteria, Calif.) or 20 μl of anti-CD61-phycoerythrin (PE) (BD Pharmingen) in 200 μl 0.01% bovine serum albumin (BSA)-PBS at 4° C. for 50 min with spinning to realize homogeneous fluorescent staining. After centrifugation (1,000 rpm, 10 min), the cell pellets thus obtained were rinsed three times with 0.01% bovine serum albumin (BSA)-PBS. Fluorescence was analyzed on a fluorescence-activated cell sorter (FACS) (Aria) using a Cellquest program. The results are given in FIGS. 7A and 7B.

CD41 and CD61, which both are receptors specifically expressed upon the differentiation of HEL cells into megakaryocytes, were observed to increase in expression rate in a day-dependent manner upon treatment with (R)-NALPCE.

### EXPERIMENTAL EXAMPLE 7

### Differentiation Depending on the Presence of (R)-NALPCE and OP9 Cell

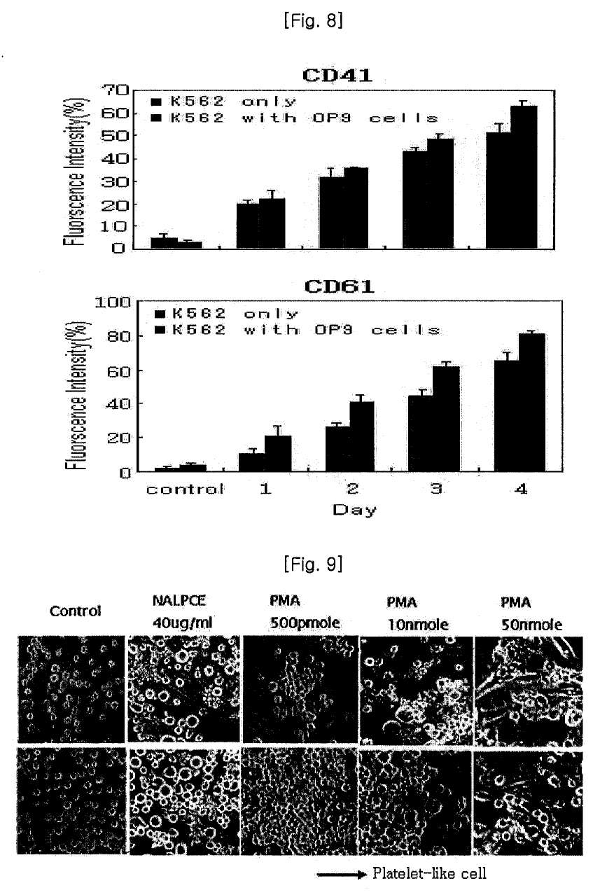

OP9 cells were aliquoted at a density of 1×10⁵ cells per well in 6-well plates and incubated for 24 hrs. After OP9 cells were uniformly attached onto the bottom of the plates, K562 cells were layered at a density of 1×10⁵ cells per well over the OP cells in the 6-well plates. Treatment with 40 μg/ml of (R)-NALPCE was conducted before incubation in a day-dependent manner for 4 days. Following centrifugation (1,000 rpm, 5 min), the cell pellets thus obtained were rinsed three times with 0.01% bovine serum albumin (BSA) in PBS. Incubation with 10 μl of anti-CD41-fluorescein-5-isothiocyanate (FITC) (Dako Chemicals, Carpenteria, Calif.) and 20 μl of anti-CD61-phycoerythrin (PE) (BD Pharmingen) in 200 μl of 0.01% bovine serum albumin (BSA)-PBS was conducted at 4° C. for 50 min so as to achieve homogeneous fluorescent staining. After centrifugation (1,000 rpm, 10 min), the fluorescent-stained cell pellets thus obtained were washed three times with 0.01% bovine serum albumin (BSA) in PBS. Fluorescence was analyzed on a fluorescence-activated cell sorter (FACS) (Aria) using a Cellquest program. The results are given in FIG. 8.

Although having the activity of inducing differentiation of K562 cells into megakaryocytes even when used alone, the compound (R)-NALPCE was found to further effectively induce K562 cells to differentiate into megakaryocytes when used in combination with OP9 cells, as demonstrated by a 15% increase in the count of CD41-positive cells.

### EXPERIMENTAL EXAMPLE 8

### Cell Differentiation Upon Treatment with (R)-NALPCE and PMA

A stromal line of OP9 cells derived from mouse calvaria was aliquoted at a density of 1×10⁵ cells in 6-well plates and incubated for 24 hrs. After the OP9 cells were uniformly attached onto the bottom of the plates, K562 cells were layered at a density of 1×10⁵ cells on the OP9 cells in 6-well plates. The cells were treated with 40 μg/ml of (R)-NALPCE, or 500 pmole, 10 nmole or 50 nmole of PMA before incubation for 5 days. They were observed under an optical microscope with 200× magnification. The results are given in FIG. 9.

As seen in FIG. 9, when K562 cells were treated with (R)-NALPCE, they increased in size in a time-dependent manner, and anucleate cells such as thrombocytes were observed. When treated with as low as 500 pmole of PMA, K562 cells did not differentiate, but proliferated, like a control. Treatment with 10 nmole of PMA allowed K562 cells to increase in size, but with morphology different from that of those treated with (R)-NALPCE. Even 5 days or more after the treatment, no morphology similar to thrombocytes was observed. As for treatment with 50 nmole of PMA, it induced K562 cells to differentiate into macrophages. In consequence, PMA could allow the expression of a lot of CD41, like (R)-NALPCE, but could not produce thrombocyte-like cells.

### EXPERIMENTAL EXAMPLE 9

### Cell Differentiation upon Treatment with (R)-NALPCE and PMA

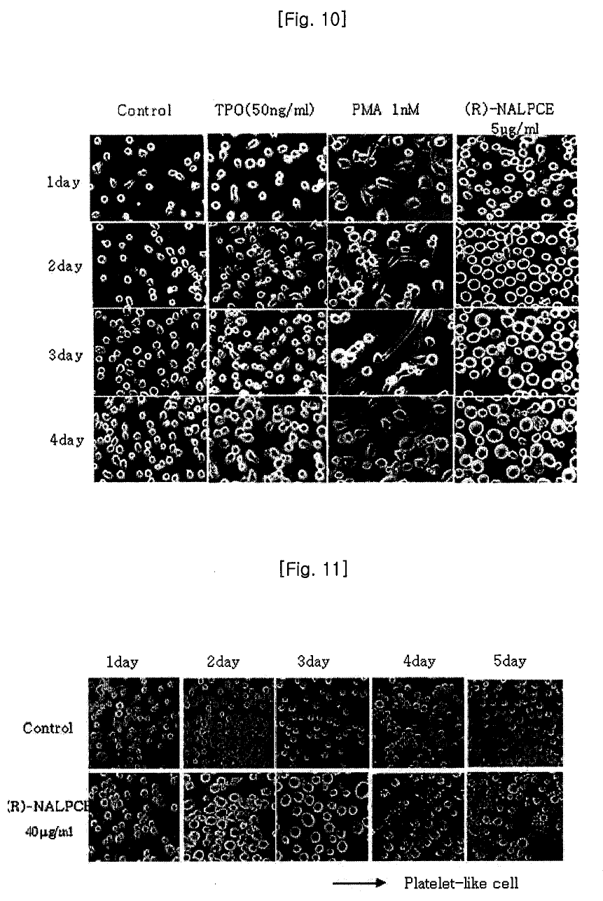

HEL cells were aliquoted at a density of 3×10⁵ cells per well in 6-well plates and treated with 5 μg/ml of (R)-NALPCE, 10 nmole of PMA, or 50 ng/ml of TPO, followed by incubation in a day-dependent manner for 5 days. They were observed under an optical microscope with 200× magnification. The results are given in FIG. 10.

As seen in FIG. 10, HEL cells, when treated with (R)-NALPCE, were observed to increase in size in a day-dependent manner. Upon treatment with PMA, however, HEL cells increased in size, but with morphology different from that upon treatment with (R)-NALPCE, and differentiated into macrophages. HEL cells did not undergo significant change after being treated with 50 ng/ml of TPO.

### EXPERIMENTAL EXAMPLE 10

### Production of Thrombocytes by (R)-NALPCE

An experiment was conducted in a manner similar to that of Example 7. OP9 cells were aliquoted at a density of 1×10⁵ cells per well in 6-well plates and incubated for 24 hrs. After OP9 cells were uniformly attached onto the bottom of the plates, K562 cells were layered at a density of 1×10⁵ cells per well over the OP cells in the 6-well plates. Treatment with 40 μg/ml of (R)-NALPCE was conducted before incubation in a day-dependent manner for 5 days. They were observed under an optical microscope with 200× magnification. The results are given in FIG. 11.

As seen in FIG. 11, K562 cells did not change in size in the absence of (R)-NALPCE, but increased in size in a time-dependent manner when treated with (R)-NALPCE. From Day 4 after the treatment, anucleate cells, such as thrombocytes, were observed.

### EXPERIMENTAL EXAMPLE 11

### Binding to Fibrinogen of Cells Differentiated by (R)-NALPCE

Bone marrow progenitor cells, such as K562 cells, have glycoprotein IIb/IIIa complexes expressed thereon at a low level, but the number of complexes increases as the cells increase in size according to differentiation into megakaryocytes. By taking advantage of the fact that fibrinogens, which play a key role in blood clotting, bind to activated glycoprotein IIb/IIIa (GP IIb/IIIa) complex, the differentiation of K562 cells into megakaryocytes by (R)-NALPCE was investigated.

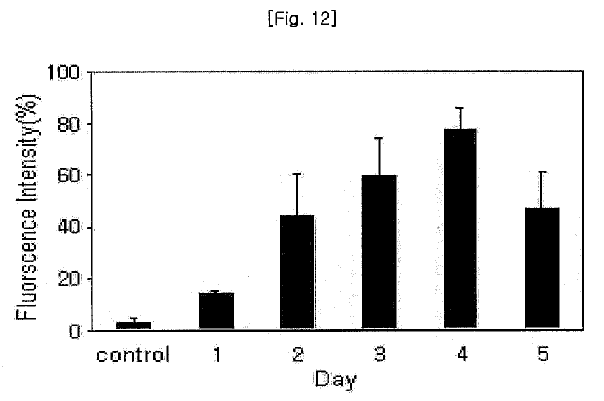

A process similar to that of Experimental Example 8 was conducted. OP9 cells were aliquoted at a density of 1×10⁵ cells in 6-well plates and incubated for 24 hrs. After the OP9 cells were uniformly attached onto the bottom of the plates, K562 cells were layered at a density of 1×10⁵ cells over the OP9 cells in 6-well plates. The cells were treated with 40 μg/ml of (R)-NALPCE before incubation in a day-dependent manner for 5 days. The (R)-NALPCE-treated K562 cells were transferred to 60 mm-dishes everyday and treated with 100 nM of PMA for 30 min to activate glycoprotein IIb/IIIa complexes. The cells thus differentiated were collected and suspended in 50 μl of PBS. The K562 cells were activated at 37° C. for 1 hr in the presence of 1 mM of MnCl₂, 50 mM of adenosine diphosphate, 50 mM of epinephrine, 1 mM of the PAR4 thrombin receptor-activating amino acid sequence AYPGFK (Peptron Inc. Korea), and 300 μg/ml of FITC-fibrinogen (Alexa-468, Molecular Probe) . Following the addition of 450 μg of PBS, the amount of fibrinogen bound to the surface of the activated K562 cells was quantitatively determined as fluorescence was analyzed on a fluorescence-activated cell sorter (FACS) (Aria) using a Cellquest program. The results are given in FIG. 12.

It is to be understood from the data of FIG. 12 that the (R)-NALPCE-induced megakaryocyte differentiation of K562 cells increases glycoprotein IIb/IIIa complexes in number as the fluorescence of the fibrinogens bound to glycoprotein IIb/IIIa complexes is found to increase in a day-dependent manner.

### EXPERIMENTAL EXAMPLE 12

### Electromicroscopic Observation of Cells (Megakaryocytes, Thrombocytes) Differentiated by (R)-NALPCE

OP9 cells were aliquoted at a density of 1×10⁵ cells in 6-well plates and incubated for 24 hrs. After the OP9 cells were uniformly attached onto the bottom of the plates, K562 cells were layered at a density of 1×10⁵ cells over the OP9 cells in 6-well plates. The cells were treated with 40 μg/ml of (R)-NALPCE before incubation for 5 days. Following centrifugation (1,000 rpm, 5 min), the cells were pre-fixed with 2.5% glutaraldehyde in PBS (pH 7.4) for 24 hrs. Thereafter, cell pellets obtained by centrifugation (4,000 rpm, 10 min) were fixed for 1 hr with 1% osmium tetraoxide in 0.1 M PBS (pH 7.4). After being washed with PBS, the post-fixed samples were dehydrated with 60, 70, 80, 90, and 95% ethanol for 10 min each and then twice with 100% ethanol for 10 min each, and embedded in epoxy resin. The embedded samples were sectioned into slices 1 μm thick using an ultramicrotome diamond knife (Richert-Jung, U.S.A.) and stained with 1% toluidine blue for optical microscopy. Separately, ultra-thin sections ranging in thickness from 60 to 70 nm were prepared and stained sequentially with 1-2% uranyl acetate and 1% lead citrate for transmission electron microscopy, in which electrons were accelerated at 75 kV using H-600 TEM (Hitachi, Japan).

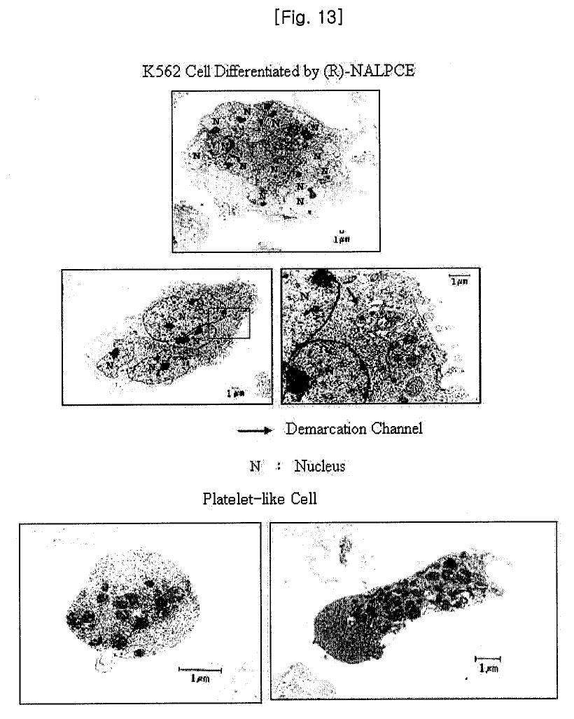

As seen in FIG. 13 providing transmission electron microscopic photographs of K562 cells treated with (R)-NALPCE, polymorphonuclear megakaryocytes having at least two nuclei were observed to form 40% or more of the resulting differentiated cells and to have demarcation channels within which platelets are held. Thus, the megakaryocytes are mature enough to release platelets. Also, the platelets released from the mature megakaryocytes have morphology similar to that of the pre-existing blood platelets.

### EXPERIMENTAL 13

### Morphology of Active Form of Platelet-Like Cells Secreted from K562 Cells Differentiated by (R)-NALPCE

OP9 cells were aliquoted at a density of 1×10⁵ cells per well in 6-well plates and incubated for 24 hrs. After the OP9 cells were uniformly attached onto the bottom of the plates, K562 cells were layered at a density of 1×10⁵ cells over the OP9 cells in the 6-well plates. The cells were treated with 40 μg/ml of (R)-NALPCE before incubation in a day-dependent manner for 5 days. On Day 5 after the treatment, a large number of anucleate platelet-like cells appeared. The platelet-like cells were separated from the megakaryocytes by centrifuging the differentiated K562 cells at 500 rpm for 20 min and further centrifuging the supernatant at 3,000 rpm for 10 min.

A morphological comparison was made between the platelet-like cells and the pre-existing blood platelets using fibrinogen-coated slides. General slides (POLY-PREP, Sigma, U.S.A.) were coated with 20 μg/ml of fibrinogen in PBS ((R)-NALPCE-treated group) or with 1% bovine serum albumin (control) in PBS, and then incubated at 37° C. for 2 hrs in a humid atmosphere. A suspension of the separated platelet-like cells and the pre-existing blood platelets in PBS was placed on the slides. After the cells adhered to the slides in 20 min, PBS was removed and 2% paraformaldehyde was placed for 10 min on the cells to fix them. Afterwards, the cells were permeabilized by treatment with 0.1% Triton X-100 for 10 min. After the removal of Triton X-100, 1 mM of MnCl₂, 50 mM of adenosine diphosphate, 50 mM of epinephrine, or 1 mM of the PAR 4 thrombin receptor activating amino acid sequence AYPGFK (Peptron Inc., Korea) in 50 μl of PBS was added onto the slides and incubated at 37° C. for 1 hr to activate the separated cells. 1 hr after incubation, the removal of the solution was followed by gently washing with PBS. A drop of a mounting solution was spotted on the slides, which were then covered with a coverslip before observation under a fluorescence microscope. For actin observation, phalloidin-fluorescein-5-isothiocyanate (phalloidin-FITC) (Alexa-468, Molecular Probe) was diluted 1:50 in PBS, applied to the slides, and incubated at 37° C. for 1 hr in a humid atmosphere. The cells thus stained were very gently washed three times with PBS and observed using a fluorescence microscope (Axiovision, Zeiss) with 400× magnification.

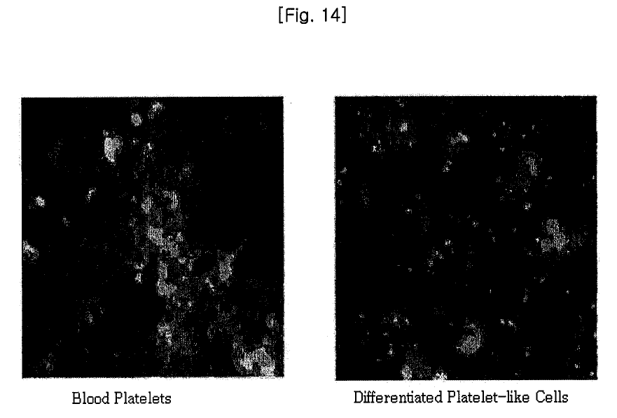

Thrombocytes can be activated by adenosine diphosphate, epinephrine, a PAR4 thrombin receptor activating amino acid sequence (AYPGFK, Peptron Inc., Korea), or collagen. As apparent from data of FIG. 14, the anucleate cells released from the K562 cells differentiated by (R)-NALPCE are activated in the same manner as the pre-existing blood platelets, and, once activated, they have cellular structures similar to those of the pre-existing blood platelets.

### INDUSTRIAL APPLICABILITY

When used in cooperation, as described hitherto, OP9 cells and (R)-NALPCE can effectively induce the differentiation of leukemia cells derived from human bone marrow into megakaryocytes and further into thrombocytes, and the thrombocytes released from the differentiated megakaryocytes are useful in the treatment of thrombocytopenia.

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced other than as specifically described.

1. A method for inducing the differentiation of leukemia cells derived from human bone marrow into megakaryocytes or thrombocytes, comprising the steps of:
(a) culturing OP9 cells;
(b) layering leukemia cells derived from human bone marrow over the OP9 cells; and
(c) incubating the cells in the presence of a compound represented by the following Chemical Formula 1.

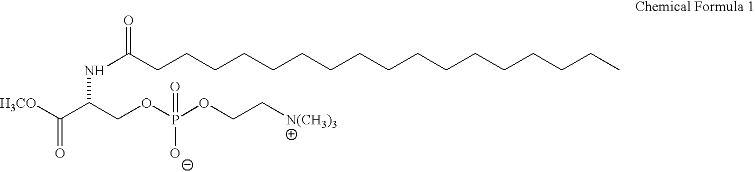

2. The method according to claim 1, wherein OP9 cells are cultured for a period ranging from 18 to 36 hours in step (a).

3. The method according to claim 1, wherein the compound of Chemical Formula 1 is used at a concentration ranging from 10 to 50 μg/ml.

4. A composition for inducing the differentiation of leukemia cells derived from human bone marrow into megakaryocytes or thrombocytes, comprising OP9 cells and a compound represented by the following Chemical Formula 1. [chemical structure]
